(19) United States
(12) Patent Application Publication
(10) Pub. No.: US 2008/0058726 A1
(43) Pub. Date: Mar. 6, 2008

(54) Methods and Apparatus Incorporating a Surface Penetration Device

(76) Inventors: Arvind Jina, San Jose, CA (US); Bee Lee Chua, Davis, CA (US); Shashi Desai, San Jose, CA (US); Ashok Parmar, Fremont, CA (US); Abdel-Nasser Kawde, Fremont, CA (US)

Correspondence Address: SHAYGLENN LLP, 2755 CAMPUS DRIVE, SUITE 210, SAN MATEO, CA, 94403, US

(21) Appl. No.: 11/468,732

(22) Filed: Aug. 30, 2006

(51) Int. Cl. A61M 5/32

(52) U.S. Cl. 604/174; 604/272

(57) ABSTRACT

A surface penetration device, methods of use and methods of manufacture thereof are provided. The device includes a substrate supporting a tapered elongate structure extending from and supported by the substrate upper surface. The elongate structure includes a distal portion, a proximal portion supported at the substrate upper surface, a lumen, and a plurality of lumen openings. A cross section of the lumen is asymmetrical and includes a fist defining dimension that is larger than a second defining dimension. The lumen extends from the lumen openings and through the elongate structures. The lumen openings are positioned proximal to the elongate structure's distal portion. In one embodiment an array of a plurality of the elongate structures are provided. In another embodiment, the elongate structures are microneedles.

### FIELD OF THE INVENTION

The present invention relates to the design and fabrication of long micro needles with small elongated through-substrate lumen. More specifically, the present invention relates to a microfabricated device for penetrating surface and accessing substances beneath the surface for biomedical applications such as transdermal drug delivery and analyte detection and monitoring.

### BACKGROUND OF THE INVENTION

A number of designs and microfabrication techniques are presently used to fabricate micro needles with through-substrate lumens. The micro needles length varies from 50 μm to more than 1 mm. The lumen size varies from 10 μm to more than 100 μm. The techniques used to fabricate the micro needles include in-plane and out-of-plane silicon micromachining, polymer molding such as SU-8 and PDMS, and thermal pulling of glass.

Current approaches to fabricating out-of-plane silicon-based micro needles with through-substrate lumens involve the use of deep etching from one side or both sides of the substrate. The shape of the lumen is typically circular or square. It is positioned either in-line with, or at an offset from the center of the micro needle. It is advantageous to control the size of the lumen opening on the micro needle, independent of the lumen etch depth, hence the length of the needle. This allows accurate positioning of the lumen opening on the micro needle's surface.

Current design and batch fabrication techniques cannot manufacture long out-of-plane micro needles with small lumen openings, and hence provide limited benefit in transdermal drug delivery and monitoring. On the other hand, long in-plane micro needles with small lumen openings cannot be manufactured in large arrays.

Current precision fabrication techniques such as laser drilling individual lumen on the micro needle are prohibitively expensive.

Hence, what is needed is a design and batch fabrication technique, which can manufacture long micro needles with small lumen openings. Although microneedle arrays are known in the art, they have certain limitations and drawbacks that result in limited efficacy and range of use in a variety of medically relevant applications.

### SUMMARY OF THE INVENTION

In general, in one aspect, the invention features methods and apparatus including a surface penetration device. The methods include methods of manufacture and use for a surface penetration device. The surface penetration device of invention includes a substrate with an upper surface and a backside, and further includes a tapered elongate structure extending from and supported by the substrate upper surface. The elongate structure includes a distal portion, a proximal portion supported at the substrate upper surface and a lumen. A cross section of the lumen is asymmetrical and comprises a first defining dimension that is larger than a second defining dimension. The elongate structure further includes a plurality of lumen openings, wherein the lumen extends from the lumen openings and through the elongate structure, and wherein at least one lumen opening comprises an elongate geometry and is positioned proximal to the elongate structure's distal portion.

Implementations of the invention can include one or more of the following features. The elongate structure can be an array of a plurality of tapered elongate structures. In one implementation, the elongate structures are microneedles. In another implementation, the lumen openings can be a proximal lumen opening and a plurality of distal lumen openings. The lumen can extend from the proximal lumen opening to at least one of the plurality of distal lumen openings. In one implementation the cross section of the lumen is a non-square or non-circular geometry. In a further implementation, the length of the lumen is greater than the width of the lumen.

The elongate structure can further include a plurality of inclined faces. In one implementation, the elongate structure includes four inclined faces. The lumen can be substantially centered within the elongate structure and oriented substantially non-parallel to each inclined face of the elongate structure. Alternatively, the lumen can be substantially centered within the elongate structure and oriented substantially parallel to at least one inclined face of the elongate structure. Where the lumen opening includes an elongate geometry, the at least one lumen opening can determined by the position and orientation of the lumen within the elongate structure.

In one embodiment, the at least one lumen opening comprises length and width dimensions wherein the width dimensions are independent of a length dimension of the elongated structures. In another embodiment, the distal portion of the elongate structure further includes at least one rounded end, wherein the radius of curvature of the at least one rounded end is smaller than a girth of the elongate structure.

In a particular embodiment, the lumen and lumen openings are sized to support capillary force for interstitial fluid or water.

In general, in another aspect, a method of accessing interstitial fluid using a surface penetration device is provided. The method can include providing a microneedle device including a substrate and one or more tapered elongate structures supported by the substrate, wherein the elongate structures each include a lumen, a plurality of lumen openings, and wherein at least one lumen opening includes an elongate geometry. A further step includes penetrating a skin surface with the elongate structures and delivering a substance to or sampling a substance associated with interstitial fluid. In one implementation, the method further includes monitoring the glucose level of the interstitial fluid wherein the sampled substance is glucose. In another implementation, wherein a substance is delivered, the delivered substance can include a drug.

In general, in another aspect, a method of manufacturing a surface penetration device is provided. The method can include providing a substrate including a front side and a backside, forming a backside opening and a lumen in the substrate, wherein the lumen extends distally from the backside opening, through the substrate, and ends proximal to the upper surface.

A cross section of the lumen can be asymmetrical and include a first defining dimension that is larger than a second defining dimension. The method further includes providing a tip protection cap to a portion of the substrate front side, wherein the cap comprises a dimension, further forming a trench in the uncapped portion of the substrate and a micro pillar in the capped portion of the substrate. The micro pillar can be sharpened to form a tapered elongate structure and at least one lumen opening with an elongate geometry and in communication with the lumen.

In one implementation, the cap dimension is smaller than the substrate. In another implementation providing a tip protection cap includes providing a plurality of tip protection caps, wherein forming a trench comprises forming a plurality of trenches and micro pillars, and wherein sharpening the micro pillars results in an array of a plurality of elongated structures.

In one implementation, formation of the backside opening, lumen, trench, micro pillar and sharpening of the micro pillar is achieved using an etching or mechanical process. In a particular embodiment, the method further includes the step of applying an etchant resistant material to the micro pillars.

In one implementation, the tapered elongate structure distal portion includes at least one rounded end and the etchant resistant material is configured to guide shaping of the tapered elongate structure, including modifying the curvature of the at least one rounded end. In a further embodiment the tapered elongate structure includes a height to proximal portion girth ratio of greater than one.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic of a top view of a microneedle layout design of the invention.

FIG. 2 is a schematic of a side view of the substrate of the invention prior to sharpening micro pillars.

FIG. 3A is a schematic of a side view of the substrate of the invention after sharpening micro pillars.

FIG. 3B is a schematic of an orthogonal view of a microneedle of the invention.

FIG. 3C is a schematic of a cross-section view of a proximal portion of a microneedle of the invention.

FIG. 3D is a schematic of a side view of a microneedle of the invention.

FIG. 3E is a schematic of a cross-section view of a proximal portion of a microneedle of the invention.

FIG. 4A is a schematic of an array of microneedles of the invention.

FIG. 4B is a schematic of an alternative array of microneedles of the invention.

FIG. 4C is a photomicrograph of an array of microneedles of the invention.

FIG. 5 is a schematic of a glucose monitoring system including the array of microneedles of the invention.

FIG. 6 is a block diagram showing steps for manufacturing the array of microneedles of the invention.

### DETAILED DESCRIPTION OF THE INVENTION

The following description is presented to enable any person skilled in the art to make and use the invention, and is provided in the context of a particular application and its requirements. Various modifications to the disclosed embodiments will be readily apparent to those skilled in the art, and the general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

The present invention involves devices and methods that enable improved sampling of substances of interest and/or improved delivery of substances (e.g., drugs) through a surface (e.g., such as the skin or outer layer of a plant, animal, or organ or part thereof) by using out-of-plane microneedles. While surface penetration in the examples discussed herein may relate to biological surfaces including cell surfaces, it is envisioned that the devices and methods described herein can be applied to any of a number of physical surfaces including but not limited to plastic, metal, cloth, fabric, paper etc.

Although a standard metal hypodermic needle is effective in piercing the outer layer of skin (e.g., stratum corneum) and accessing the tissue and blood vessels beneath it for monitoring, sampling or delivery, interfacing a standard sized needle with a silicon microchip is a challenging problem of scale. Larger needles also create discomfort and safety issues when used in various research and/or treatment settings. A discussion of these issues is disclosed in U.S. patent application Ser. No. 10/995,570, filed Nov. 22, 2004, the disclosure of which is incorporated herein by reference in its entirety.

Great reduction in needle size creates a number of problems. Microneedles of various heights, (e.g. for humans, with a shank height in the range of about 150-350 microns) have been proposed for various monitoring or delivery applications. However, even very sharp microneedles often fail to pierce skin or other biological surfaces due to the soft underlying tissue and the elastic nature of the skin that can result in bending and folding around the needle tip to a depth greater than the needle shank height. If the microneedles are made taller, but of the same diameter, they tend to bend (buckle) at pressures less than that required to pierce the desired surface, e.g., the tough stratum corneum of many areas of the human skin.

Due to the very small needle openings of many proposed hollow microneedles, tips may also clog. This is especially problematic if the opening is at or very near the apex (or tip) of the needle. These problems are solved with specific embodiments of microneedles and/or systems and/or methods of the present invention as described herein.

Mammalian skin can be categorized into two distinct layers. In humans, the surface layer (epidermis) is primarily made up of 100 micron-2 mm thick layer of epithelial cells. The underlying layer (dermis) typically includes a 1.1 mm layer of connective tissue. Skin thickness varies at different locations of the body as well as epidermal/dermal proportions. For example, the epidermis is extremely thick (400-600 microns) on the palms of the hand and soles of the feet, whereas the dermis is thickest near the upper back (1.1 mm). The epidermis itself lacks blood vessels and draws nourishment by diffusion from vessels located 200-500 microns away in the underlying dermis. Although free nerve endings are present in the epidermis, experiments have shown that breaching the epidermis generally causes no or very little pain.

A minimally invasive method for sampling biological fluids is a prerequisite to performing either periodic or continuous monitoring of physiological systems. Blood and cellular interstitial fluid (ISF) contain important metabolic and immunological biomolecules. The time varying concentrations of these molecules are important indicators of various states of health and disease. The transdermal sampling of small volumes of blood for glucose concentration measurement is part of the daily routine for many diabetic patients to monitor and control the symptoms of their disease. The use of microtechnology to reduce the size of needles to minimize discomfort is a rapidly developing arena of investigation for the transcutaneous delivery of drugs. However, despite the rapid advances in lab-on-a chip technology that can separate, concentrate and analyze biological indicators, the ability to deliver minute biological samples to the microchip for analysis requires further development. A few demonstrations of transdermal biological fluid extraction and analysis using microfabricated devices and have been reported, using microneedles and thermal ablation.

Although a standard metal hypodermic needle is quite effective in piercing the stratum corneum, and accessing the tissue and blood vessels beneath it, interfacing a standard sized needle with a silicon microchip is a challenging problem of scale. Creating a micro-miniature version of this structure presents not only a less invasive and less painful extraction method, but also enables the integration of the sample delivery device with analysis system. However, great reduction in size creates some new problems. Even experimentation using very sharp microneedles with a tip radius of curvature less than 2 microns often fail to pierce skin because of the soft underlying tissue and the elastic nature of the skin, which can bend and fold around the needle tip to a depth greater than the needle shank height. If the needles are made taller, but of the same diameter, they tend to bend (buckle) at pressures less than that required to pierce the tough stratum corneum of the skin. Another problem is tip clogging, due to the very small needle openings. This is especially problematic if the opening is at the apex of the needle, because it is forced into the skin.

A number of techniques for forming needles from semiconductors or other materials that can be similarly etched or molded have been discussed. For example, U.S. Pat. No. 6,406,638, by inventors Boris Stoeber and Dorian Liepmann and assigned to The Regents of the University of California (Oakland, Calif.), entitled “Method of forming vertical, hollow needles within a semiconductor substrate, and needles formed thereby” discusses forming a needle by anisotropically etching a channel into the back side of a semiconductor substrate with the front side of the semiconductor substrate then isotropically etched to form a vertical axial surface surrounding the channel. Co-assigned U.S. Pat. No. 5,928,207, invented by Albert P. Pisano and Kyle S. Lebouitz, and entitled “Microneedle with Isotropically Etched Tip, and Method of Fabricating such a Device”, uses isotropic etching to form microneedles in the horizontal plane of a semiconductor substrate. Another semiconductor fabrication technique for forming needles is discussed by Neil H. Talbot, Christopher G. Keller, and Albert P. Pisano, in their U.S. Pat. No. 6,106,951, entitled “Apparatus and Method for Fabricating Needles Via Conformal Deposition in Two-Piece Molds”. This technology forms a needle via conformal deposition within a horizontally-oriented chamber defined by a two-piece mold.

### REFERENCES

A number of references may be considered relevant to the present invention or provide background material or details regarding methods known in the art that may have relevance to specific embodiments of the invention. The following as well as any publications cited herein are hereby incorporated by reference for all purposes.
[1] S. Henry, D. V. McAllister, M. G. Allen, M. R. Prausnitz, Microfabricated microneedles: a novel approach to transdermal drug delivery, J. Pharm. Sci. 87 (1998) 922-925.
[2] E. T. Lagally, C. A. Emrich, R. A. Mathies, Fully integrated PCRcapillary electrophoresis microsystem for DNA analysis, Lab Chip 1 (2001) 102-107.
[2] J. Liu, K. Tseng, B. Garcia, C. B. Lebrilla, E. Mukerjee, S. Collins, R. Smith, Electrophoresis separation in open microchannels. A method for coupling electrophoresis with MALDI-MS, Anal. Chem. 73 (2001) 2147-2151.
[4] N. Szita, J. Dual, and R. Buser. An actuation coupling system for a fast and low volume micropipetting device with integrated sensors, presented at ACTUATOR 2000, 7th International Conference on New Actuators and International Exhibition on Smart Actuators and Drive Systems, Conference Proceedings. MESSE BREMEN GMBH, Bremen, Germany, 2000, pp. 228-231.
[5] S. R. Visuri, K. Ness, J. Dzenitis, B. Benett, K. Bettencourt, J. Hamilton, K. Fisher, and P. Krulevitch. Microfluidic tools for biological sample preparation, presented at 2nd Annual International IEEE-EMBS Special Topic Conference on Microtechnologies in Medicine and Biology, Proceedings (Cat. No. 02EX578). IEEE, Piscataway, N.J., USA, 2002, pp. 556-559.
[6] W. H. Smart, K. Subramanian, The use of silicon microfabrication technology in painless blood glucose monitoring, Diabetes Technol. Therapeutics 2 (2000) 549-559.
[7] J. G. E. Gardeniers, J. W. Berenschot, M. J. de Boer, Y. Yeshurun, M. Hefetz, R. van't Oever, and A. van den Berg, Silicon micromachined hollow microneedles for transdermal liquid transfer, presented at MEMS 2002 IEEE International Conference, Fifteenth IEEE International Conference on Micro Electro Mechanical Systems (Cat. No. 02CH37266) Technical Digest. MEMS 2002 IEEE International Conference, Fifteenth IEEE International Conference on Micro Electro Mechanical Systems, Las Vegas, Nev., USA, 2002.
[8] P. Griss, G. Stemme. Novel, side opened out-of-plane microneedles for microfluidic transdermal interfacing, presented at MEMS 2002 IEEE International Conference, Fifteenth IEEE International Conference on Micro Electro Mechanical Systems (Cat. No. 02CH37266) Technical Digest. MEMS 2002 IEEE International Conference. Fifteenth IEEE International Conference on Micro Electro Mechanical Systems, Las Vegas, Nev., USA, 2002.
[9] S. Chandrasekaran, J. Brazzle, and A. B. Frazier, Surface Micromachined Metallic Microneedles, J. Microelectromechan. Syst. 12 (2003) 281-288.

Surface penetration apparatuses, methods of use and methods of manufacturing thereof are provided. Devices of the invention can include one or a plurality of elongate structures (e.g., microneedles) that can be arranged in one or more regular or irregular intervals on a supporting substrate. Generally, each elongate structure has one or more through substrate lumen, although it is envisioned that some of the microneedles (when arranged in a plurality) need not include such lumens. A lumen of the device generally includes a cross section that is asymmetrical in nature and includes a first defining dimension that is larger than a second defining dimension. At least one lumen opening of the elongate structure includes an elongate geometry positioned proximally to a distal part of the elongate structure. In one embodiment, as a result of the microneedle sharpening process a plurality of small elongated lumen openings are revealed on the surface of the microneedle. Uses for the microneedle arrays of the invention include penetrating a surface (e.g., the skin) and accessing the substances beneath the surface. As such applications include but are not limited to accessing interstitial fluids for physiological monitoring and delivery of agents including but not limited to drugs. Although the examples discussed herein generally relate to biological substance accessing, sampling and/or delivery, it is envisioned that any form of substance can be used with the devices and methods described herein. Substances envisioned include but are not limited to fluids, a gas or gases, solids or semisolids.

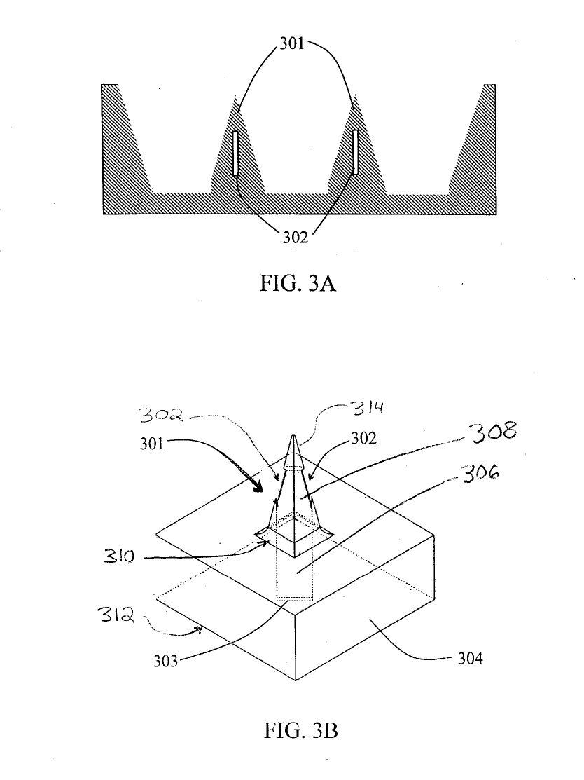

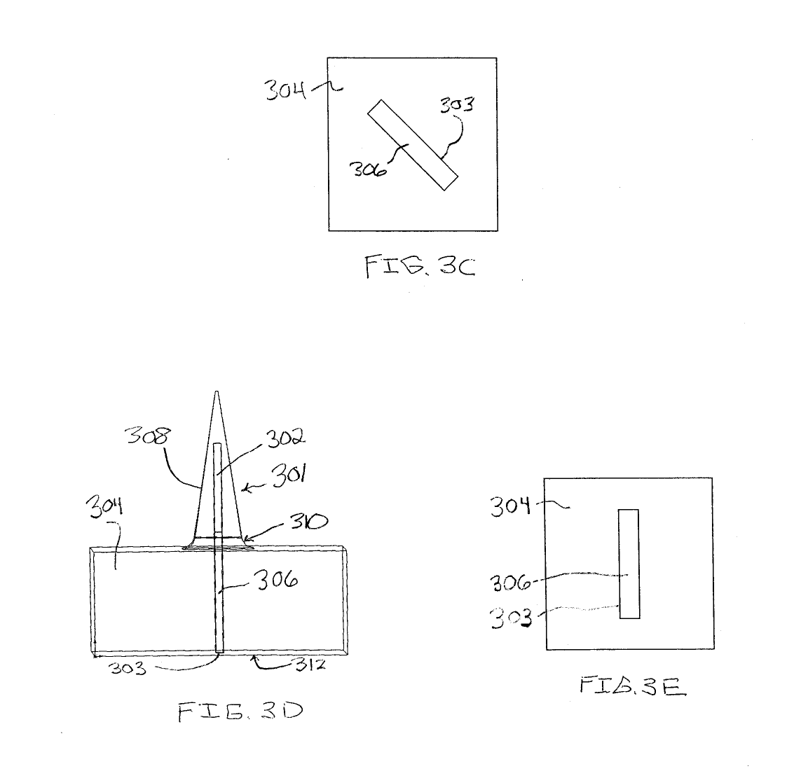

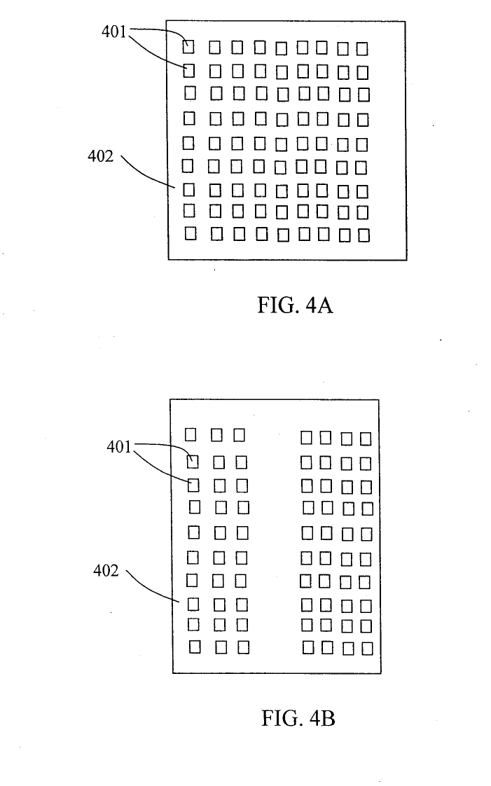

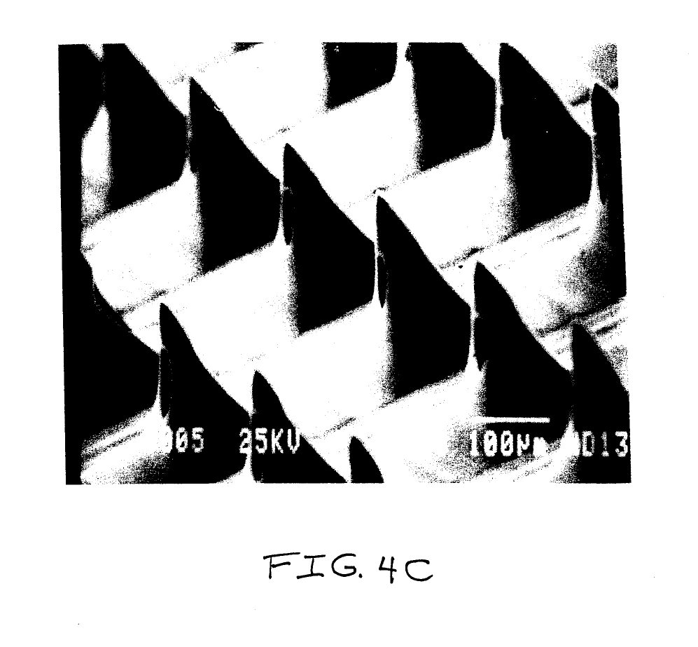

As shown in FIGS. 3B, 3D and 4A-C, in general, the micro array needle device of the invention can include a substrate, and an arrayed plurality of tapered elongate structures (e.g., microneedles) supported by the substrate. The elongate structures include a proximal end where connected to the substrate and a distal portion. The elongate structures include a lumen and a plurality of lumen openings wherein the lumen openings are positioned on the elongate structure some distance away from the distal portion of the elongate structures.

As shown in FIGS. 4A-C, in certain embodiments the device includes a plurality of microneedles arranged to form microneedle arrays. FIG. 4A, illustrates a square microneedle array in accordance with an embodiment of the invention. The micro needles 401 can be arranged at regular or irregular intervals on the substrate 402. It is envisioned that in some embodiments lumens may be selectively absent from some microneedles (not shown).

FIG. 4B illustrates a non-square silicon microneedle array in accordance with an embodiment of the invention. The microneedles 401 can be arranged at regular or non-regular intervals on the substrate 402. In the example shown in FIG. 4B, the array of microneedles 401 and the shape of the substrate 402 are both non-square. The arrays of microneedles 401 can also be arranged discontinuously on the substrate 402 in any desired fashion, including but not limited to the arrangement illustrated in FIG. 4B where a gap exists between array sections.

FIG. 4C is a photomicrograph of another example of a microneedle array of one embodiment of the invention.

The device of the invention including the substrate and the elongate structure can be made up of one or more structurally hard material. Suitable materials include but are not limited to glass, plastic, silicon, germanium, minerals (e.g. quartz), semiconducting materials (e.g. silicon, germanium, etc.), ceramics, metals, ceramic, polymer and plastic.

In general, the elongate structures in the various embodiments of the invention are forms of microneedles. The microneedles can range in length from one to twenty millimeters. In one embodiment the microneedles are less than one millimeter in length. The microneedles can have an overall shape that includes but is not limited to a cone- or multiple sided pyramid-shape. In one embodiment, the microneedles include a plurality of inclined faces or surfaces. As illustrated in FIG. 3B, in a particular embodiment the microneedles include four inclined faces.

The microneedle arrays described herein can include one or more internally disposed lumen. The lumens can be shaped in any of a number of ways using well known micro-fabrication techniques. The lumens can be a bore. The cross section of the lumens can include any of a number of shapes including but not limited to round, oblong, square, rectangular, ellipsoid or any multisided shape. In one exemplary embodiment the lumen(s) are shaped as a slot extending lengthwise along at least a portion of the elongate member. As illustrated in FIGS. 3B-E, the lumen 306 can be slot-shaped and include a rectangular cross-section. In one embodiment, as illustrated in FIG. 3B, a slot-shaped lumen 306 includes length and width dimensions wherein the length of the slot is greater than the width of the slot.

FIGS. 3B-E show two exemplary lumen orientations within the device. In FIGS. 3D and 3E, the lumen 306 is substantially centered within a microneedle 301 and oriented substantially parallel to at least one inclined face 308 of the microneedle 301. FIGS. 3B and 3C illustrate a lumen 306 within the microneedle 301 wherein the lumen 306 is substantially centered within the microneedle 301 and oriented substantially non-parallel to each inclined face 308 of the microneedle 301. It is envisioned that where an odd number of faces are employed in the microneedle shaping, such a lumen could be parallel to some faces and non-parallel with respect to other faces (not shown).

Although the illustrations in FIGS. 3B-E show lumen 306 centered within a microneedle 301, it is envisioned that the lumen could advantageously be offset in the microneedle 301, for example, to accommodate multiple lumens 306 for increased material transfer and/or increasing the number of lumen openings 302.

Advantageously, as shown in FIGS. 3A-B and 3D, the lumen 306 shape can provide an elongated geometry to the lumen openings 302 of the microneedles 301. It can be understood that in one embodiment the lumen openings comprise length and width dimensions wherein the width dimensions are independent of a length dimension of the elongated structures. It is envisioned that the lumen openings can be positioned on the microneedles independently of a length dimension and a proximal portion girth dimension of the elongate structures. In other words, the positioning and dimensions of the openings along the microneedle can be varied as desired. The shaping of the microneedle and the shape of the lumens being just two controlling factors for positioning and dimensioning the lumen openings.

It is envisioned that the lumens of the invention can include variable dimensions within desired portions of the device including the substrate and the microneedle. For example, the lumens could be dimensioned to be smaller in the substrate or a portion of the substrate than in the microneedle (not shown). Alternatively, the lumens could be dimensioned to be smaller in all or a portion of the microneedle than in the substrate (not shown).

It is envisioned that a lumen can extend from the lumen opening to the proximal portion of the microneedle. In the embodiments illustrated in FIGS. 3B and 3D, the lumen 306 is shown to further extend from the proximal portion 310, through the substrate 304 to the backside opening 303 at the substrate backside 312.

The distal portions of the microneedles of the invention can include one or more rounded ends or tips. The rounded ends can include a radius of curvature such that the round end is smaller in diameter than the girth of the microneedle. The rounded ends can be shaped in any of a number of ways well known in the field of microneedles. In one exemplary embodiment as shown in FIG. 3B, the rounded end 314 is cone-shaped. It is envisioned that the rounded end could be blunted or sharpened as desired. In a case where the rounded end is sharp, it can be designed as a surface penetration point configured to penetrate a surface. The surface penetrated can be a visco-elastic surface including but not limited to skin, tissue an organ. The sharp rounded end can be configured to penetrate other surfaces including plant tissues and cultured animal or plant cells. It is envisioned that such an embodiment would be useful, for example, in tattooing.

In general, in another aspect a method of manufacturing a surface penetration device (e.g., a microneedle array device) is provided. As shown in block diagram form in FIG. 6, the manufacturing process 600 includes the steps: prepare a substrate 602, form backside opening and lumen 604, provide microneedle tip protection cap 606, form trenches and micro pillars 608, sharpen micro pillars 620 and form lumen openings 612. More specifically, the method includes providing a substrate including a front side and a backside. A backside opening and a lumen are formed in the substrate wherein the lumen extends distally from the backside opening, through the substrate and ending near the front side. A microneedle tip protection cap is provided to portions of the substrate front side. Trenches are formed in the uncapped portions of the substrate and micro pillars are formed in the capped portions of the substrate. The micro pillars are sharpened to form tapered elongate structure (e.g., microneedles) with a plurality of lumen openings in communication with the lumen.

Formation of the various structural elements of the device can be achieved using etching or mechanical processes. In some embodiments the elements are produced using a fabrication method including but not limited to dry plasma etching, wet aqueous etching, molding, stamping, water jet milling, solid particles ablation and photon or electron beam milling. Where the elongate structures are microneedles, using a fabrication method the microneedles can be manufactured to include a narrower distal portion than proximal portion. Additionally, the microneedles can be fabricated to include a height (or length) to proximal portion (e.g., base) girth ratio of greater than one.

Fabrication of the rounded ends can be achieved by, for example, dry plasma etching or wet etching in addition to any of the fabrication methods disclosed herein.

Fabrication of the lumens from the substrate can be achieved by, for example, a fabrication method including dry plasma etching, wet aqueous etching, water jet drilling, solid particles ablation and photon or electron beam drilling. The lumens can be configured as a conduit for a liquid or gaseous substance. The liquid substance can include but is not limited to saline solution, aqueous chemicals and organic fluids. The lumens and lumen openings can be sized to support capillary force and capillary flow. It is further envisioned that flow in the lumens can controlled by pumping or pressure regulation.

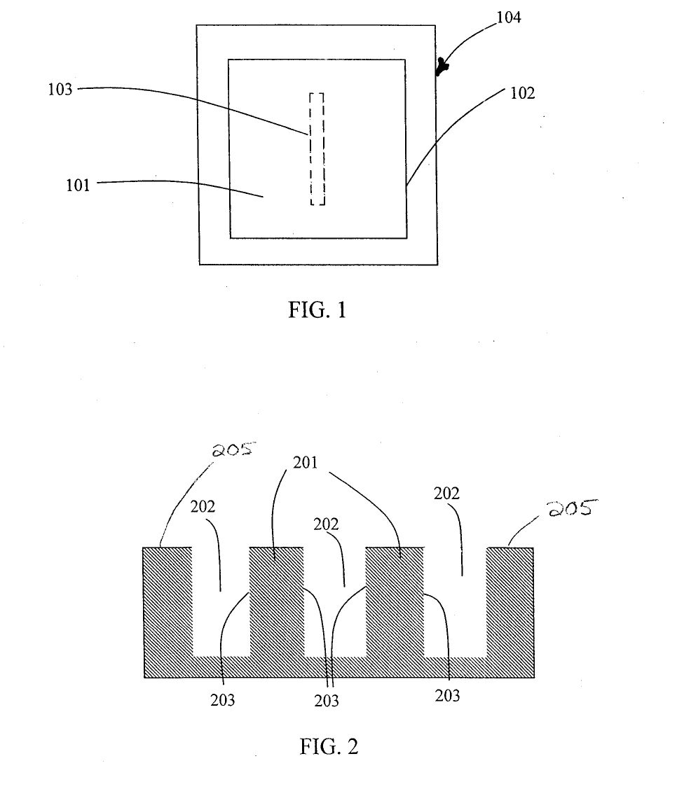

FIG. 1 shows a top view of a microneedle layout design in accordance with an embodiment of the present invention. The structural component of the microneedle is fabricated from bio-inert or bio-compatible substrate 101. One possible material for bio-inert or bio-compatible substrate 101 is silicon. A micro needle tip protection cap 102 can be deposited on substrate 101. The cap 102 can be an etchant resistant material including but not limited to HNA, TMAH, EDP, KOH, SF6, XeF2, HF, CHF3 and CF4. In one embodiment the material for the microneedle tip protection cap is silicon nitride. A silicon nitride cap on the front side of the substrate can define a top face of the silicon micro pillar. In a further embodiment the etchant resistant material is configured to guide shaping of the micro pillar, including modifying the curvature of the rounded ends. In a further variation, the silicon nitride cap may be smaller than the actual top face of the silicon micro pillar.

FIG. 1 further shows one embodiment of patterning the backside lumen opening 103 on the backside of the substrate 101. The width of lumen opening 103 can range from 1 μm to 200 μm. In one embodiment the range of width is between 1 μm to 50 μm. The length of the lumen opening 103 is typically longer than that of its width by at least 10% of the width's dimension. Where the lumen opening 103 are slot-shaped as illustrated in FIG. 1, the slot shape enables deep through substrate etching to form long microneedles without enlarging the width of the opening on the micro needle's surface. This provides advantages over square or circular cross-section-shaped lumens.

As shown in FIG. 1, the size of the microneedle tip protection cap 102 typically can define the shape of the micro pillar 104. As shown, the size of a given dimension (e.g., cap width or the length of a cap side) of the microneedle tip protection cap 102 can be smaller than the top face of the micro pillar 104. The size of a given dimension of the microneedle tip protection cap and micro pillar top face can range from 1 μm to 1 mm. In one embodiment the size range of the cap and micro pillar top face is from 10 μm to 500 μm. The shape of the microneedle tip protection cap 102 and micro pillar 104 is typically, but not limited to, square-shaped.

As further shown in FIG. 1, the position and alignment of the lumen opening 103 are typically, but not limited to, centered to the microneedle tip protection cap 102. The orientation of the lumen opening 103 is typically, but not limited to, 90 or 45 degrees to the edge of the micro pillar 104.

FIG. 2 presents a side view of a silicon micro pillar 201 prior to sharpening in accordance with an embodiment of the invention. The micro pillar can be formed by creating trenches 202, for example, in a successive manner. Typically the trenches are formed by wet etching, dry etching or mechanical methods such as dicing saws. As shown in FIG. 2, the sidewalls 203 of the micro pillar 201 can be, but need not necessarily be, vertical.

FIG. 3A presents a side view of a silicon microneedle 301 after sharpening of a micro pillar (see FIG. 2) in accordance with an embodiment of the invention. The sharpening is performed by selectively removing more micro pillar material near the top of the eventual silicon microneedle. As shown in FIG. 3A, an elongated lumen opening 302 can be revealed on the surface of the silicon micro needle 301 after the sharpening of a micro pillar. Advantageously, as shown in FIGS. 3A and 3D, the elongated lumen opening 302 can be relatively long and narrow in comparison to the bored hole lumen openings of older microneedle designs.

FIG. 3B presents an orthogonal view of a silicon microneedle after sharpening in accordance with an embodiment of the present invention. The multiple small elongated lumen openings 302 are a direct result of sharpening a micro pillar (see FIG. 2) and the deep etching of the lumen 306 by way of the backside lumen opening 303 of the substrate 304. Advantageously, as shown in FIG. 3B, a single etched lumen 306 can provide for multiple elongated lumen openings 302 upon sharpening. Although the manufacture of the lumen openings described herein has focused on a process wherein the lumen is formed before the sharpening process, it is envisioned that the process of forming the lumen could follow the preparation of the microneedle feature or in any desired succession of etching and or mechanical processing steps as described herein.

In general in another aspect, methods of accessing interstitial fluids are provided. In one method of accessing an interstitial fluid a microneedle device is provided including a substrate and one or more tapered elongate structures (e.g., microneedles) supported by the substrate. Each elongate structure includes a lumen, a proximal lumen opening and a plurality of distal lumen openings. The method further includes penetrating a skin surface with the elongate structure and delivering a substance or sampling a substance from interstitial fluid beneath the surface through the distal lumens openings. This method is particularly useful for applications such as physiological monitoring and drug delivery.

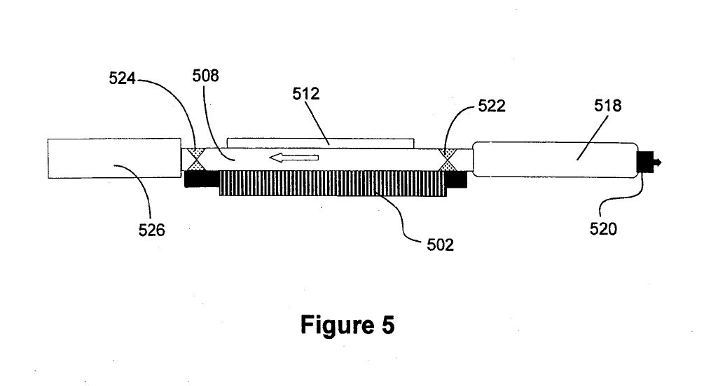

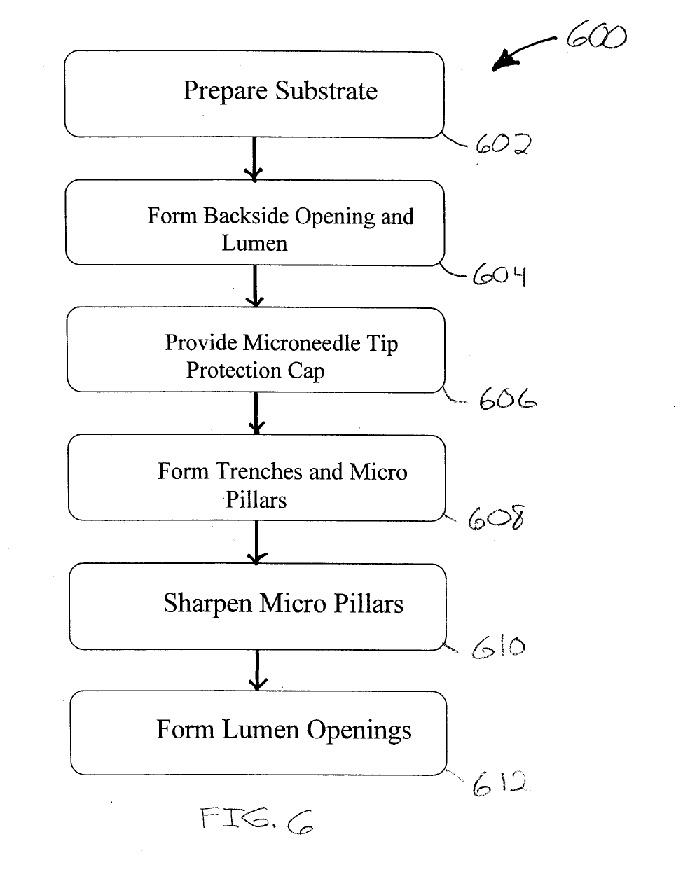

One useful application of this method is use in combination with a glucose monitoring device as shown in FIG. 5. As illustrated, the microneedle array 502 of the invention can be included as a part of the glucose monitoring device 500. In use, the microneedles can be used penetrate the stratum corneum, for example, of the skin, in order to access the interstitial fluid for glucose level monitoring (not shown). Details of a suitable glucose monitoring device can be found in U.S. application Ser. No. 11/277,731, filed Mar. 28, 2006, the disclosure of which is incorporated herein by reference in its entirety.

As shown in FIG. 5, the glucose monitoring device 500 can include the microneedle array 502 and a glucose sensor 512 in fluid communication with a sensing area in channel 508. In the illustrated embodiment, actuator 520 is on the side of sensing fluid reservoir 518, and the waste reservoir 526 is expandable. Operation of actuator 520 sends sensing fluid from reservoir 518 through one way flap valve 522 into the sensing area in channel 508 and forces sensing fluid within channel 508 through flap valve 524 into the expandable waste reservoir 526.

In the embodiment of FIG. 5 (and potentially other embodiments), the starting amount of sensing fluid in the calibration reservoir 518 is about 1.0 ml or less, and operation of the sensing fluid actuator 520 sends a few microliters (e.g., 10 μL) of sensing fluid into channel 508. Recalibrating the device three times a day for seven days will use less than 250 μL of sensing fluid.

Thus, a microneedle-based system according to specific embodiments of the invention can be employed as an effective glucose monitor using a microneedle array for accessing interstitial fluid. The glucose monitor can be attached to a skin location (for example, with a self-adhesive, medical tape, a band, etc.) by the patient himself without an assisted insertion procedure.

Another useful application of the method of accessing interstitial fluid is for the delivery of one or more drugs to the interstitial fluid, for example from a reservoir. A variety of details relating to drug delivery using a microneedle array device is disclosed in U.S. Pat. No. 6,611,707 to Prausnitz et al., the disclosure of which is incorporated by reference herein in its entirety. In one embodiment, delivery of the drug from a reservoir in communication with the microneedles of the array is initiated by applying a force, such as by pressing the top of the reservoir, to cause the reservoir contents (i.e. a drug containing composition) to flow out through the microneedles.

Delivery also can be initiated by opening a mechanical gate or valve interposed between a reservoir outlet and a microneedle inlet. For example, a thin film or plate can be slid or peeled away from the back of the substrate of the microneedle array.

In an alternate embodiment, delivery is initiated by changing the physical or chemical properties of the drug composition and/or of a barrier material. For example, the barrier can be a porous membrane having a porosity that can be selectably altered to permit flow, or the drug composition can be selected to change from a solid or semi-solid state to a fluid state, for example as the temperature is increased from ambient to that of body temperature. Such a drug composition can be prepared, for example, by combining the drug with a biodegradable polymeric material.

Delivery also can be initiated by activating an osmotic pump, as described, for example, in U.S. Pat. No. 4,320,758 to Eckenhoff, which has been adapted to the substrate of the microneedle device. For example, the reservoir/osmotic pump includes an inner flexible bag that holds the drug charge, an intermediate layer of an osmotically effective solute composition, such as an inorganic salt, that encapsulates the bag, and an outer shape-retaining membrane that is at least in part permeable to water and that encapsulates both the layer of osmotically effective solute composition and the bag. In operation, the bag filled with the fluid drug compositions is exposed to an aqueous environment, so that water is imbibed from the environment by the osmotically effective solute through the membrane into the space between the inner flexible bag and the membrane. Since the bag is flexible and the membrane is rigid, the imbibed water squeezes the bag inwardly, thereby displacing drug out the microneedles.

In an alternate embodiment, delivery is initiated by opening the pathway between the reservoir and the microneedles of the array and simply allowing the drug to be delivered by diffusion, that is, a passive process.

In a one embodiment, the microneedle device includes a feedback means so that the user can (1) determine whether delivery has been initiated; and/or (2) confirm that the reservoir has been emptied, that is delivery complete. Representative feedback means include a sound, a color (change) indicator, or a change in the shape of a deformable reservoir. In another embodiment, the feedback for completion of delivery is simply that the reservoir is pressed flat against the back of the substrate and cannot be further deformed.

The user of the microneedle device typically can determine if the microneedles have been properly inserted into the skin or other tissue through visual or tactile means, that is assessing whether the substrate has been pressed essentially flush to the tissue surface. For example, if a puddle of liquid drug composition appears near the device, then the user may infer that the microneedles are not fully inserted, suggesting that the device needs to be reapplied. The liquid drug compositions can include a coloring agent to enhance the visual feedback.

In a more complex embodiment, an electrical or chemical measurement is adapted to provide the feedback. For example, penetration can be determined by measuring a change in electrical resistance at the skin or other tissue, or a pH change. Alternately, needle-to-needle electrical resistance can be measured. In a preferred embodiment, the microneedle device includes a disposable cartridge containing the microneedles. In these devices, an LED (e.g. green light/red light) or liquid crystal display can be provided with the reusable portion of the device.

The microneedle device must be capable of transporting drug across or into the tissue at a useful rate. For example, the microneedle device must be capable of delivering drug at a rate sufficient to be therapeutically useful. The rate of delivery of the drug composition can be controlled by altering one or more of several design variables. For example, the amount of material flowing through the needles can be controlled by manipulating the effective hydrodynamic conductivity (the volumetric through-capacity) of a single device array, for example, by using more or fewer microneedles, by increasing or decreasing the number or dimensions of the lumen openings in the microneedles, or by filling at least some of the microneedle lumens with a diffusion-limiting material. In order to simplify the manufacturing process it is envisioned the needle design could be limited to two or three “sizes” of microneedle arrays to accommodate, for example small, medium, and large volumetric flows, for which the delivery rate is controlled by other means.

Other means for controlling the rate of delivery include varying the driving force applied to the drug composition in a reservoir. For example, in passive diffusion systems, the concentration of drug in the reservoir can be increased to increase the rate of mass transfer. In active systems, for example, the pressure applied to the reservoir can be varied, such as by varying the spring constant or number of springs or elastic bands.

In either active or passive systems, the barrier material can be selected to provide a particular rate of diffusion for the drug molecules being delivered through the barrier at the needle inlet.

Essentially any drug can be delivered using the microneedle devices described herein. As used herein, the term “drug” refers to an agent which possesses therapeutic, prophylactic, enhancement or diagnostic properties in vivo, for example when administered to a plant or an animal, including mammals, such as humans. Examples of suitable therapeutic and/or prophylactic active agents include proteins, such as hormones, antigens, and growth factors; nucleic acids, such as antisense molecules; and smaller molecules, such as antibiotics, steroids, decongestants, neuroactive agents, anesthetics, and sedatives. Examples of suitable diagnostic agents include radioactive isotopes and radioopaque agents, metals, gases, labels including chromatographic, fluorescent or enzymatic labels.

The drug can be or include a peptide, protein, carbohydrate (including monosaccharides, oligosaccharides, and polysaccharides), nucleoprotein, mucoprotein, lipoprotein, glycoprotein, nucleic acid molecules (including any form of DNA such as cDNA, RNA, or a fragment thereof, oligonucleotides, and genes), nucleotide, nucleoside, lipid, biologically active organic or inorganic molecules, or combination thereof.

The amount of drug can be selected by one of skill in the art, based, for example on the particular drug, the desired effect of the drug at the planned release levels, and the time span over which the drug should be released.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A surface penetration device comprising:
a substrate comprising an upper surface and a backside;
a tapered elongate structure extending from and supported by the substrate upper surface, the elongate structure comprising a distal portion, a proximal portion supported at the substrate upper surface, a lumen, wherein a cross section of the lumen is asymmetrical and comprises a first defining dimension that is larger than a second defining dimension, and a plurality of lumen openings, wherein the lumen extends from the lumen openings and through the elongate structure, and wherein at least one lumen opening comprises an elongate geometry and is positioned proximal to the elongate structure's distal portion.

2. The device of claim 1, wherein the elongate structure comprises an array of a plurality of tapered elongate structures.

3. The device of claim 2, wherein the elongate structures comprise microneedles.

4. The device of claim 1, wherein the lumen openings comprise a proximal lumen opening and a plurality of distal lumen openings.

5. The device of claim 4, wherein the lumen extends from the proximal lumen opening to at least one of the plurality of distal lumen openings.

6. The device of claim 5, wherein the cross section of the lumen comprises a non-square or non-circular geometry.

7. The device of claim 1, wherein the length of the lumen is greater than the width of the lumen.

8. The device of claim 1, wherein the elongate structure further comprises a plurality of inclined faces.

9. The device of claim 8, wherein the elongate structure comprises four inclined faces.

10. The device of claim 9, wherein the lumen is substantially centered within the elongate structure and oriented substantially non-parallel to each inclined face of the elongate structure.

11. The device of claim 8, wherein the lumen is substantially centered within the elongate structure and oriented substantially parallel to at least one inclined face of the elongate structure.

12. The device of claim 1, wherein the elongate geometry of the at least one lumen opening is determined by the position and orientation of the lumen within the elongate structure.

13. The device of claim 12, wherein the at least one lumen opening comprises length and width dimensions wherein the width dimensions are independent of a length dimension of the elongated structures.

14. The device of claim 1, wherein the distal portion of the elongate structure further comprises at least one rounded end, wherein the radius of curvature of the at least one rounded end is smaller than a girth of the elongate structure.

15. The device of claim 1, wherein the lumen and lumen openings are sized to support capillary force for interstitial fluid or water.

16. A method of accessing interstitial fluid comprising:
providing a microneedle device comprising a substrate and one or more tapered elongate structures supported by the substrate, wherein the elongate structures each comprise a lumen, a plurality of lumen openings, and wherein at least one lumen opening comprises an elongate geometry;
penetrating a skin surface with the elongate structures; and
delivering a substance to or sampling a substance associated with interstitial fluid.

17. The method of claim 16, further comprising monitoring the glucose level of the interstitial fluid wherein the sampled substance is glucose.

18. The method of claim 16, wherein the delivered substance comprises a drug.

19. A method of manufacturing a surface penetration device comprising:
providing a substrate comprising front side and a backside;
forming a backside opening and a lumen in the substrate, wherein the lumen extends distally from the backside opening, through the substrate, and ends proximal to the upper surface, and wherein a cross section of the lumen is asymmetrical and comprises a first defining dimension that is larger than a second defining dimension;
providing a tip protection cap to a portion of the substrate front side, wherein the cap comprises a dimension;
forming a trench in the uncapped portion of the substrate and a micro pillar in the capped portion of the substrate;
sharpening the micro pillar to form a tapered elongate structure and at least one lumen opening comprising an elongate geometry in communication with the lumen.

20. The method of claim 19, wherein the cap dimension is smaller than the substrate.

21. The method of claim 19, wherein providing a tip protection cap comprises providing a plurality of tip protection caps, wherein forming a trench comprises forming a plurality of trenches and micro pillars, and wherein sharpening the micro pillars results in an array of a plurality of elongated structures.

22. The method of claim 19, wherein formation of the backside opening, lumen, trench, micro pillar and sharpening of the micro pillar is achieved using an etching or mechanical process.

23. The method of claim 19, further comprising the step of applying an etchant resistant material to the micro pillars.

24. The method of claim 19, wherein the tapered elongate structure distal portion comprises at least one rounded end and wherein the etchant resistant material is configured to guide shaping of the tapered elongate structure, including modifying the curvature of the at least one rounded end.

25. The method of claim 19, wherein the tapered elongate structure comprises a height to proximal portion girth ratio of greater than one.
